(19) United States
(12) Patent Application Publication
(10) Pub. No.: US 2025/0201424 A1
(43) Pub. Date: Jun. 19, 2025

(54) A METHOD FOR DETERMINING A PHYSIOLOGICAL AGE OF A SUBJECT

(71) Applicants: INSERM [INSTITUT NATIONAL DE LA SANTÉ ET DE LA RECHERCHE MÉDICALE], Paris (FR); CENTRE NATIONAL DE LA RECHERCHE SCIENTIFIQUE, Paris (FR); UNIVERSITE TOULOUSE CAPITOLE, Toulouse (FR); CENTRE HOSPITALIER UNIVERSITAIRE DE TOULOUSE, Toulouse (FR); UNIVERSITE TOULOUSE III - PAUL SABATIER, Toulouse (FR); ETABLISSEMENT FRANCAIS DU SANG, La Plaine Saint Denis (FR)

(72) Inventors: Louis CASTEILLA, Toulouse (FR); Isabelle ADER, Toulouse (FR); Philippe KEMOUN, Toulouse (FR); Julien ALIGON, Toulouse Cedex 9 (FR); Paul MONSARRAT, Toulouse (FR); Sylvain CUSSAT-BLANC, Toulouse (FR); David BERNARD, Toulouse (FR); Emmanuel DOUMARD, Toulouse (FR); Luc PENICAUD, Toulouse (FR)

(21) Appl. No.: 18/849,701

(22) PCT Filed: Mar. 23, 2023
(86) PCT No.: PCT/EP2023/057449

§ 371 Date: Sep. 23, 2024

(30) Foreign Application Priority Data
Mar. 24, 2022 (EP) 22305353.9

(51) Int. Cl. G16H 50/50

(52) U.S. Cl. CPC G16H 50/50

(57) ABSTRACT

It is disclosed a computer-implemented method for determining a physiological age of a subject, comprising applying, on a set of values comprising at least values of biological variables relative to the subject, a trained model configured to predict the chronological age of a subject based on the set of values, to obtain a predicted age of the subject, wherein the physiological age corresponds to said predicted age.

### TECHNICAL FIELD

The present disclosure relates to a computer-implemented method for determining the physiological age of a subject and detecting premature ageing of said subject.

### BACKGROUND OF THE INVENTION

The ageing of populations with its socio-economic consequences has become a major issue for all societies. While for a long time the main goal of aging investigations was to increase longevity, present investigations are focusing on healthy aging and the need to consider the organism as a whole in order to maintain the intrinsic capacity and the crucial functions. This turn was accompanied by the emergence of geroscience paradigm considering that age is the main risk factor shared by all chronic diseases and for which age-related molecular dysfunctions are causative of the function decline (Kennedy, B. K. et al. Geroscience: Linking Aging to Chronic Disease. Cell 159, 709-713 (2014)). However, whereas the first perspective is quite fully-integrated, most of geroscience investigations are conducted at cell or even molecular scales.

At the organism level, ageing results from multifactorial process dysfunctions related to a large number of mostly interdependent mechanisms that a single variable cannot describe. Their accumulation on health trajectory is very variable between people with the same chronological age (Li, Q. et al. Homeostatic dysregulation proceeds in parallel in multiple physiological systems. Aging Cell 14, 1103-1112 (2015)). To capture these dysfunctions, usually appearing subtly even before any clinical signs, it is therefore critical to early monitor a physiological age at the individual scale. The aim is not solely to provide the most appropriate personalized recommendations and interventions to achieve healthy aging but also to assess the efficacy of anti-aging therapies.

Recently, deep learning approaches have been undertaken to predict chronological age, with the limit of the explainability of the model (Putin, E. et al. Deep biomarkers of human aging: Application of deep neural networks to biomarker development. Aging 8, 1021-1033 (2016), Cohen, A. A., Morissette-Thomas, V., Ferrucci, L. & Fried, L. P. Deep biomarkers of aging are population-dependent. Aging 8, 2253-2255 (2016)). However explainability is an essential requirement both for the acceptability, and the applicability for medical uses but also, to generate physio-pathological hypotheses.

### SUMMARY OF THE INVENTION

In this context, an aim of the present disclosure is to provide an improved method for assessing physiological age of a subject and detecting premature ageing.

Another aim of the present disclosure is to propose an explainable machine learning framework.

Accordingly, a computer-implemented method for determining a physiological age of a subject is disclosed, comprising applying, on a set of values comprising at least values of biological variables relative to the subject, a trained model configured to predict the chronological age of a subject based on the set of values, to obtain a predicted age of the subject, wherein the physiological age corresponds to said predicted age.

In embodiments, the method further comprises comparing the physiological age of the subject with the chronological age of the subject, wherein a positive difference between the physiological age of the subject and the chronological age is indicative of premature ageing of the subject.

In embodiments, the method further comprises comparing the physiological age of the subject with a reference age corresponding to a mean age predicted by the trained model on a population of the same chronological age as the subject, wherein a positive difference between the physiological age of the subject and the reference age is indicative of premature ageing of the subject.

In embodiments, the method further comprises comparing the physiological age of the subject with a reference age corresponding to a mean age predicted by the trained model on a reference population, and when the physiological age of the subject differs from the reference age, identifying the biological variables most contributing to the difference. The reference population is a population of individuals having the same chronological age as the individual. The reference population may also be a population of individuals ranging on a chronological age span of at least 50 years.

In embodiments, identifying the biological variables most contributing to the difference comprises determining SHAP values associated to each of the biological variables and identifying the SHAP values having highest absolute value.

In embodiments, the method further comprises comparing at least one value of a biological variable most contributing to the difference, to a reference value of said biological variable for the same chronological age.

The reference value of a biological variable for a given chronological age may be determined as a mean value of the biological variable among a plurality of individuals of said given chronological age for which said biological variable does not contribute to a difference between the predicted age and the chronological age. The reference value of a biological variable for a given chronological age may also be determined as a mean value of the biological variable among a plurality of individuals of said chronological age, which predicted age is inferior or equal to said chronological age. In embodiments, the method further comprises determining an ageing profile of the subject among a plurality of pre-established ageing profiles, based on the identified biological or physiological values most contributing to the difference.

In embodiments, the plurality of pre-established ageing profiles are determined by:
predicting the chronological age of a plurality of individuals of a population by implementing the trained model, wherein the population comprises for each of a plurality of chronological ages, a plurality of individuals,
determining at least a mean predicted age of the population,
determining, for a plurality of individuals of the population, the SHAP values of the biological variables most contributing to a difference between the predicted age for the individual and the mean predicted age,
performing clustering on the SHAP values to obtain a finite number of clusters, wherein each cluster corresponds to an ageing profile.

In embodiments, the method comprises determining a mean predicted age for each of a plurality of chronological ages of the population, determining the SHAP values of the biological variables most contributing to a difference between the predicted age for the individual and a mean predicted age determined for the chronological age of the individual, and wherein the clustering is performed on said SHAP values.

In embodiments, the trained model is an XGboost model with custom loss function being a function of chronological age.

In embodiments, the biological variables comprise at least a plurality among the following variables:
Glycohemoglobin,
Creatinine in urine,
Cholesterol,
Alanine transaminase (ALT),
Mean cell volume,
Aspartate Transferase (AST),
Blood urea nitrogen,
Gamma-glutamyl transferase (GGT).

According to another objects, it is disclosed a computer-program product comprising code instructions for implementing a method according to the description above, when the instructions are executed by a processor.

According to another object, a computing system is disclosed, comprising:
a processor,
a non-transitory computer-readable medium storing program code that is executable by the processor,
wherein the processor is configured for executing the program code to perform operations comprising applying, on a set of values of biological variables relative to the subject, a trained model configured to predict the chronological age of a subject based on the set of values, to obtain a predicted age of the subject, wherein the predicted age of the subject corresponds to a physiological age.

In embodiments, the processor is further configured to compute a difference between the physiological age of the subject and a reference age corresponding to a mean age predicted by the trained model on a population, the population comprising a plurality of individuals of the same chronological age as the subject, or a plurality of individuals of various chronological ages, ranging on a chronological age span of at least 50 years.

In embodiments, the processor is communicatively coupled via a data network to a client system, and is configured to receive the set of values of biological variables relative to the subject from the client system and to return to the client system the physiological age of the subject, or a difference between the physiological age of the subject and a reference age corresponding to a mean age predicted by the trained model on a population.

In embodiments, if the computed difference is different from zero, the processor is further configured to compute SHAP values associated to each of the biological variables and identifying the SHAP values having highest absolute value, said SHAP values corresponding to biological variables most contributing to the computed difference.

In embodiments, the processor is further configured to generate graphical data representing the SHAP values having highest absolute value, wherein the SHAP values contributing to increasing the age predicted by the model with respect to the reference age are represented in a first color and the SHAP values contributing to decreasing the age predicted by the model with respect to the reference age are represented in a second color.

In embodiments, the computing system further comprises a memory, and the processor is further configured to:
compute, for each individual composing a population comprising, for a plurality of chronological ages, a plurality of individuals, a predicted age of the individual, based on a set of values of the biological variables relative to the individual,
compute, for a plurality of chronological ages of individuals of the population, mean values of each biological variables for the individuals of said chronological age and for whom the predicted age equals the chronological age, and store said means values in the memory, and
to compute a difference between:
   at least one value of a biological variable most contributing to the difference between the predicted age of the subject with the reference age, and
   the reference value of said biological variable for the same chronological age.

### DESCRIPTION OF THE DRAWINGS

Other features and advantages of the invention will be apparent from the following detailed description given by way of non-limiting example, with reference to the accompanying drawings, in which:

FIG. 1 schematically represents the main steps of a method according to an embodiment,

FIG. 2 schematically represents a computing device according to an embodiment,

FIGS. 3a and 3b represent the performance of an XGBoost model for predicting chronological age respectively over a training and validation dataset, and FIGS. 3c and 3d represent the performance of an XGBoost model with a custom loss gradient function as a function of chronological age respectively over a training and validation dataset.

FIG. 4 is a chart representing the relative importance of the most important 20 variables in physiological age without contextualization.

FIG. 5 is a series of contextualized partial dependence plots for a plurality of biological variables. Each dot represents an individual, its grey level representing chronological age. On x-axis is the real value of the variable, while on y-axis is the SHAP value given to this individual for this variable.

FIGS. 6a and 6b represent a clustering of contextualized SHAP values computed on the NHANES study, and for each cluster the mean SHAP values of the most important biological variables.

FIG. 7 represents an exemplary display of a personal result comprising the most important contextualized SHAP values contributing to the difference between a physiological age and a predicted age.

### DETAILED DESCRIPTION OF EMBODIMENTS

With reference to the drawings, a computer-implemented method for determining the physiological age of a subject will now be described.

### Computing System

This method for determining physiological age of a subject may be implemented by a computing system 1 schematically shown in FIG. 2, comprising at least one processor 10, which may include one or more Computer processing unit(s) CPU, and/or Graphical Processing Unit(s) GPU, and a non-transitory computer-readable medium 11 storing program code that is executable by the processor, to implement the method described below.

The computing system 1 may also comprise at least one memory 12 storing a trained model configured for predicting the chronological age of a subject based on a plurality of biological variables. The memory 12 may be the same or be distinct from the non-transitory computer-readable medium 11 storing the program code. The memory may for instance be random-access memory (RAM), magnetic hard disk, solid-state disk, optical disk, electronic memory or any type of computer-readable storage medium. The memory 12 may also store other reference data obtained by application of the trained model on a reference population, and used as reference in below-detailed steps of the method. For instance, the memory 12 may store a mean age predicted by the model over a reference population comprising, for a plurality of chronological ages, a plurality of individuals.

The memory may also store, for each of a plurality of chronological ages:
a mean age predicted by the model over a reference population comprising a plurality of individuals of said chronological age, and
reference values of a plurality of biological variables for said chronological age.

In embodiments, the computing system 1 may be communicatively coupled to a client system via a data network 3, for instance a wireless network. The client system may be a computing system located at medical premises such as an hospital, a lab, a medical office. One of the computing system 1 and the client system 2 may comprise a screen 4 for displaying relevant data obtained through implementation of the method.

The same or a distinct computing system also comprising at least one processor, and non-transitory computer-readable medium 11 may also be used for training the model for predicting the chronological age of a subject, on a reference database 13.

### Method for Determining Physiological Age and Detecting Premature Ageing of a Subject

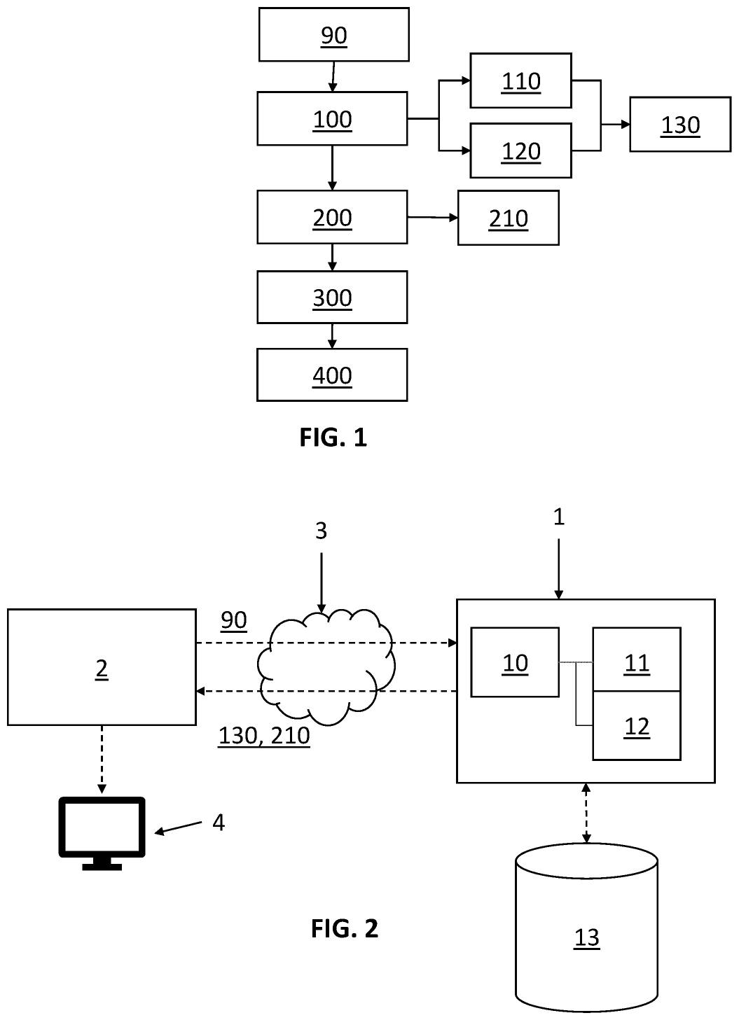

With reference to FIG. 1, a method for determining physiological age of a subject may comprise a preliminary step 90 of receiving, for a considered subject, a set of values of biological variables relative to the subject.

In embodiments, the biological variables are laboratory available variables, i.e. variables that may be obtained within a laboratory, for instance by blood analysis, urine analysis, saliva analysis, other biological fluid analysis, or direct measurement on the subject during clinical examination. In embodiments, the method may further comprise receiving, in addition to the biological variables, socio-economic variables or socio-demographic variables relative to the subject.

The biological variables may comprise at least one variable, wherein the at least one variable is glycohemoglobin.

The biological variables may comprise at least one variable, preferably a plurality, such as at least five, or all the variables among the following group:
Glycohemoglobin,
Creatinine in urine,
Cholesterol,
Alanine transaminase (ALT),
Mean cell volume,
Aspartate Transferase (AST),
Blood urea nitrogen,
Gamma-glutamyl transferase (GGT).

In embodiments, the biological variables may further comprise at least one additional variable, preferably a plurality, such as at least five, or all the variables among the following group:
Phosphorus,
Triglycerides,
Albuminemia,
Serum glucose,
Red cell distribution width,
Serum folate,
Creatinine,
Alkaline phosphatase (ALP),
Hematocrit,
Albuminuria,
Osmolality,
C-reactive protein,
Lymphocyte number.

In embodiments, the biological variables may further comprise at least one additional variable, preferably a plurality, such as at least five, ten, or all of the following variables:
mean corpuscular hemoglobin concentration (MCHC)
mean cell hemoglobin
Monocyte number
Monocyte percent
Red blood cell count
Folate, RBC
Ferritin dosage
Direct HDL-cholesterol
Basophils number
Basophils percent
Bicarbonate
Hematrocrit
Total bilirubin,
Potassium
Segmented neutrophils num
Segmented neutrophils percent
Sodium
Total calcium
Total protein
Uric acid
White blood cell count

The skilled person may refer to the NHANES laboratory methods, for instance the NHANES 2017-2020 Laboratory methods for methods for assessing each of the above biological variable.

The values of the biological or physiological variables may have been acquired from the subject and stored in a memory. The step of receiving the set of values may then comprise receiving the data through a data network or accessing to the memory in which they are stored for further processing. The step of receiving the set of values may also comprise the computing system 1 receiving said set of values from the client system 2 over the data network. The set of values may be transferred in encrypted manner or via a secure channel.

The method then comprises applying 100, on the set of values of the biological variables, a trained model configured for predicting, from said set of values, a chronological age of the subject, in order to obtain a predicted age for the subject. Said predicted age, being determined based on a set of values of biological variables, corresponds to a physiological age of a subject, which may be equal to the chronological age of the subject, or may also be inferior, or superior, to the chronological age of the subject. The latter case corresponds to a premature ageing of the subject since it implies that the physiology of the subject is older than its chronological age.

The method may thus comprise comparing 110 the predicted age of the subject with its chronological age and inferring, if the difference between the physiological age and the chronological age is positive, a premature ageing of the subject and an increased risk of developing chronic diseases, such as diabetes, coronary heart diseases, or kidney diseases.

In other embodiments, the method may comprise comparing 120 the predicted age of the subject with a reference age (which may be stored in the memory 12) corresponding to a mean age predicted by the trained model on a population of the same chronological age as the subject, and inferring, if the difference between the predicted age of the subject and the reference age is positive, a premature ageing of the subject and an increased risk of developing chronic diseases, such as diabetes, coronary heart diseases, kidney diseases.

The computing system may return to the client system 2 during a substep 130 the physiological age obtained for the subject and/or the difference between the physiological age and the reference age.

In embodiments, the physiological age obtained for a subject can be monitored at different times to follow the evolution of the physiological age of said subject. The evolution can be natural and regularly monitored to follow the evolution of a subject's health status (e.g. for detecting deleterious abnormalities and to undertake investigations about their causes in order to prevent or cure) or to study the influence of a parameter on ageing (e.g. treatments, anti-aging treatments, infections, chronic diseases, treatments of chronic diseases, diets, physical or moral stress). In particular, a deleterious abnormality is detected when the difference between the predicted age of the subject and the reference age is positive. Accordingly, in embodiments, the physiological age of a subject is calculated at least two times in order to follow the evolution of the physiological age of said subject.

### Training of the Prediction Model

The model is preliminary trained by supervised learning on a training dataset comprising, for a plurality of individuals of a population, the chronological age of each individual and values of an initial set of biological variables. The initial set of biological variables may for instance comprise part or all the above recited biological variables.

In embodiments, the initial set of biological variables comprises at least 10 variables, and preferably at least 20 variables.

The population preferably comprises individuals of chronological ages covering a wide age span, preferably of at least 50 years, with no major gender imbalance across age groups. The population may comprise more than 1000 individuals, preferably more than 10,000 individuals.

The training dataset is divided between a training subset (about 80%) and a validation subset (about 20%). The training of the model is performed to minimize the Mean Absolute Error (MAE) between the age predicted by the model and the chronological age of a subject.

The trained model is preferably an XGboost model, in which a custom objective function is introduced in order to correct the gradient used by the model to correct its error at the next iteration, using a normalization per age, as follows:

$${grad}_{i} = ( {\hat{y}}_{i} - {y}_{i} ) * \vert \frac{\frac{{\sum }_{j ∈ age ( i )} ( {\hat{y}}_{j} - {y}_{j} )}{\vert age ( i ) \vert}}{\frac{{\sum }_{k = 1}^{N} ( {\hat{y}}_{k} - {y}_{k} )}{N}} \vert$$

Where gradᵢ is the gradient to be calculated for the i^{th} individual, ŷₖ is the prediction of the model for a given iteration, y is the chronological age, age (i) represents all individuals that display the same age as the i^{th} individual, and N is the total number of individuals.

Such custom loss function as a function of chronological age allows moderating a bias of the model to predict younger and older, respectively old and young people. Furthermore, the choice of an XGboost model enables to manage missing data, and enables explainability of the model.

The training of the model may also include eliminating variables of the initial set whose contribution is not statistically greater than chance using a feature selection algorithm, for instance a GrootCV algorithm.

Additionally, Recursive Feature Elimination (RFE) may be implemented to remove the variables having the smallest contribution and which removal does not impair the quality of the model. In that case, the set of values of biological variables used for determining the physiological age of a subject comprises one value per biological variable retained at the end of said feature selection.

Back to FIG. 1, once the physiological age of the subject is determined, or when a positive difference has been computed between the physiological age of the subject and its chronological age, or between the physiological age of the subject and the mean predicted age for a population of individuals having the same chronological age, the method may further comprise determining 200 the contribution of each variable on the age predicted by the model, and identifying the biological variables most contributing to the difference. This step may comprise identifying a predetermined number of variables most contributing to the difference for instance ten or less, for instance five variables.

This can be done by computing the SHAP values for all biological variables used for the determination of the physiological age. SHAP stands for Shapley Additive exPlanations, and SHAP values were initially proposed by Lundberg, Scott et al. in «Consistent individualized feature attribution for tree ensembles», 2019.

The sum of the SHAP values for all biological variables of the model represents the individual deviation from a reference.

In a first embodiment, the reference is the mean age predicted by the model over the entire dataset. In the experiment detailed below in which the dataset is the NHANES dataset, the mean predicted age over the population is 39.9 years. Accordingly, for a given subject, the physiological age is the mean age predicted by the model over the entire dataset plus the sum of all SHAP values of respectively all the biological variables.

In a second embodiment, the reference is the mean age predicted by the model over a subpart of the dataset comprising only individuals of the same chronological age as the individual. In that case, for a given subject, the physiological age is the mean age predicted by the model for a population comprising only individuals of the same chronological age plus the sum of all SHAP values of respectively all the biological variables. In this case, the SHAP values are denoted as contextualized. The sum of the contextualized SHAP values, hereinafter denoted “iCAD”, thus represents the difference between the physiological age of the subject and a mean physiological age of a population of the same chronological age. A positive sum corresponds to a premature ageing of the subject and an increased risk of mortality.

In both embodiments, the method may comprise determining 200 the SHAP values associated to each of the biological variables and identifying those having highest absolute value, in particular the positive SHAP values having highest values, since they correspond to the biological variables most contributing in an increased physiological age with respect to the reference.

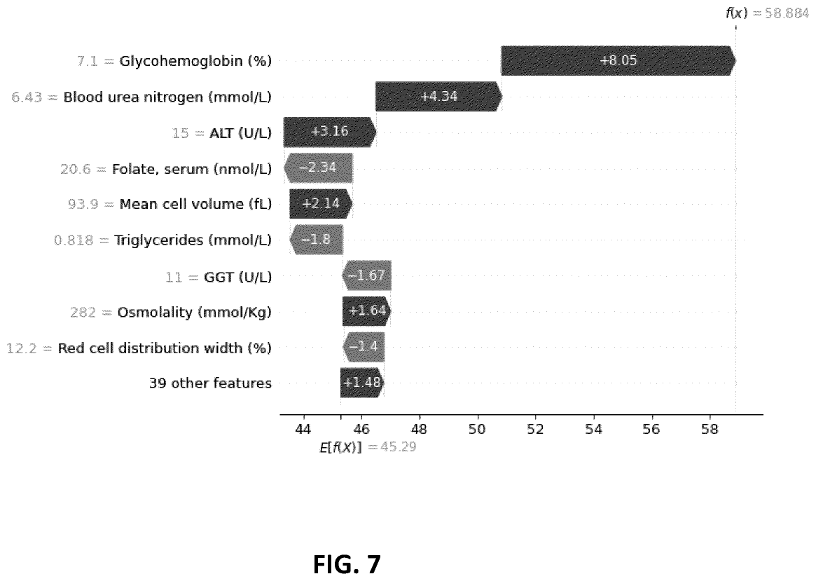

With reference to FIG. 7, the computing system 1 may generate during a step 210 graphical data to be sent to the client device 2 and displayed by the screen 4, the graphical data representing the SHAP values explaining the difference between the physiological age predicted for the subject (in the figure f(x)=58.884 years) and the reference age, which in FIG. 7 is the mean predicted age for a population of individuals of the same chronological age of the subject (E[f(X)]=45.29 years). The display of the SHAP values may comprise a chart where the abscissae represent the age and the ordinates represent the biological variables most contributing to the difference between the physiological age and the reference and their corresponding SHAP values, preferably by increasing order of importance by bottom to top. Each SHAP value may be represented by an arrow which length is at scale with the abscissae axis, where positive SHAP values are shown in a first color and negative SHAP values are shown in a second color. Also, the direction of the arrow is determined according to the sign of the SHAP values since negative SHAP values tend to lower the predicted age and positive SHAP values tend to increase the predicted age.

Also, once the biological variables most contributing to the difference between the physiological age and the reference have been identified, the subject may be submitted to regular surveillance of at least one of the variables most contributing to the difference.

In embodiments, when the biological variables most contributing to the difference between the physiological age and the reference age have been identified, their corresponding value for the subject may be compared during a step 300 with reference values of said biological variables for the same chronological age as the individual.

Reference values per biological variable and per physiological age can also be established preliminarily to implementing the method for determining physiological age of subjects, using the prediction model and its training dataset, and may be stored in the memory 12.

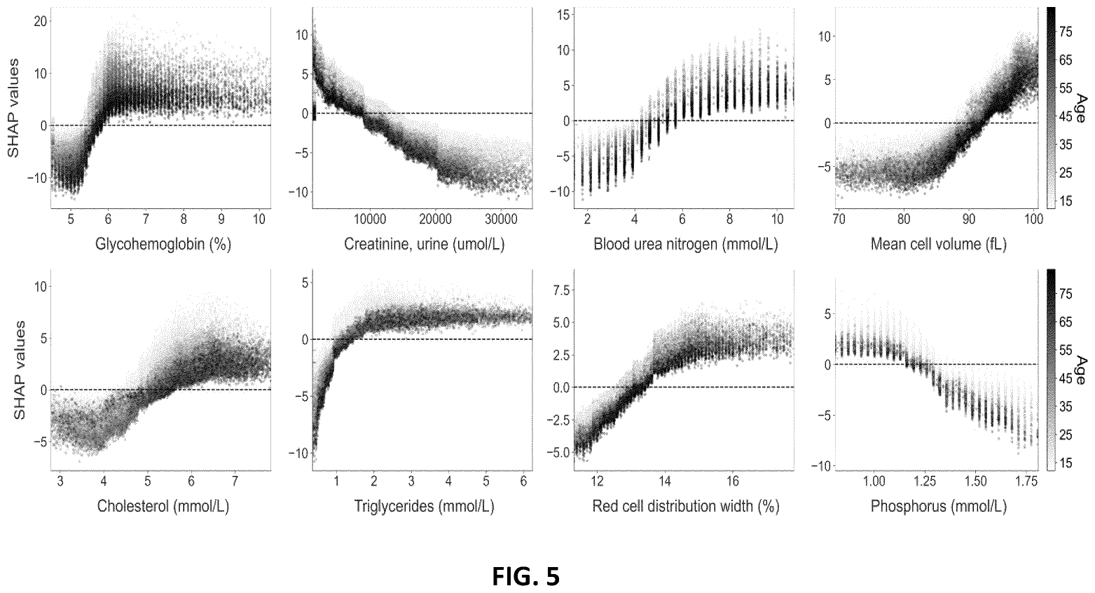

With reference to FIG. 5, are shown contextualized partial dependence plots for a plurality of biological variables including glycohemoglobin, urine creatinine, blood urea nitrogen, mean cell volume, cholesterol, triglycerides, red cell distribution width and phosphorus. Each plots displayed a plurality of dots, where each dot represents one person, and the abscissae represents the value of the corresponding biological variable, and the ordinates represent the contextualized SHAP value of said biological variable. The grey level of a dot represents the chronological age of the person. One can thus notice, according to the grey levels, that the value of a biological variable for which the SHAP value equals 0 varies according to age.

Accordingly, a chronological-age reference value for each biological variable can be determined as the mean value of the biological variable for which the corresponding SHAP value of the biological variable is zero, i.e. the biological variable does not contribute to a difference between the predicted age and the chronological age.

In another embodiment, a chronological-age reference value for each biological variable can be determined as a mean value of the biological variable among a plurality of individuals of said chronological age, for whom the predicted age is inferior or equal to said chronological age.

In embodiments, when the biological variables most contributing to the difference between the physiological age and the reference age have been identified, the method may also comprise determining 400 from said biological variables an ageing profile of the subject, from a plurality of pre-established clusters where each cluster corresponds to an ageing profile, and the clusters are established based on the biological variables most contributing to the difference between the physiological age predicted by the model and a common reference, for a population comprising a plurality of individuals covering a plurality of chronological ages. Preferably the population comprises a plurality of individuals of each of a plurality of chronological ages over an age span of at least 50 years.

More specifically, the clusters may be established by:
implementing the trained model for predicting the chronological age of a plurality of individuals of the population, thereby obtaining a physiological age of each individual,
determining at least a mean predicted age over the population, which may be a single mean predicted age over the whole population, or which may comprise for each of a plurality of chronological ages, a predicted age of a subset of individuals of the population of said chronological age,
determining, for a plurality or all the individuals of the population, the SHAP values of the biological variables most contributing to a difference between the predicted age for the individual and the mean predicted age; this step may comprise the determination for instance of the 10 or 20 highest SHAP values in absolute value,
and performing a clustering on the SHAP values obtained for the plurality of individuals.

The clustering may be performed by applying a clustering algorithm on the SHAP values, such as an agglomerative clustering algorithm, for instance a ward algorithm and Euclidean distance for linkage.

The method may further comprise generating a graphical representation of the obtained clusters, which may comprise applying an algorithm for reducing the dimensions of the SHAP values and displaying a 2D representation of the clusters by associating each dot corresponding to a cluster with a respective color. The reduction of dimensions may for instance be performed by UPAM (Uniform Manifold Approximation and Projection) or Principal Component Analysis.

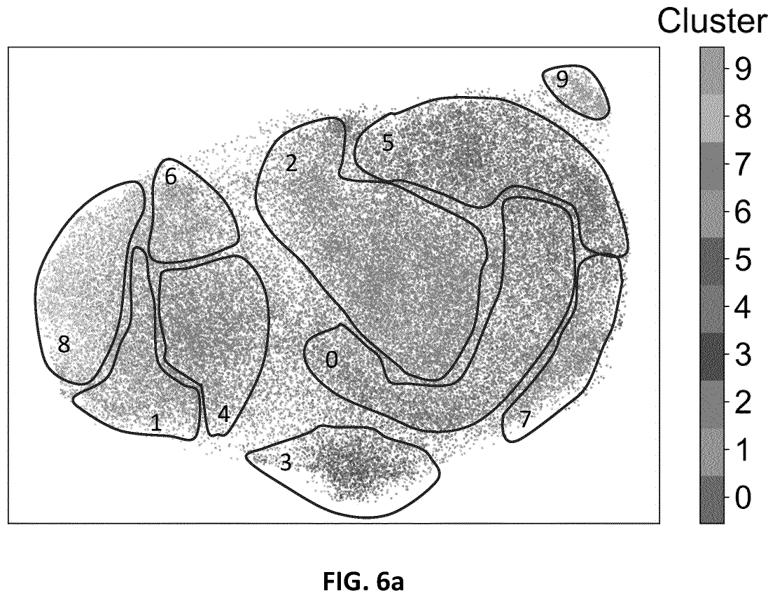

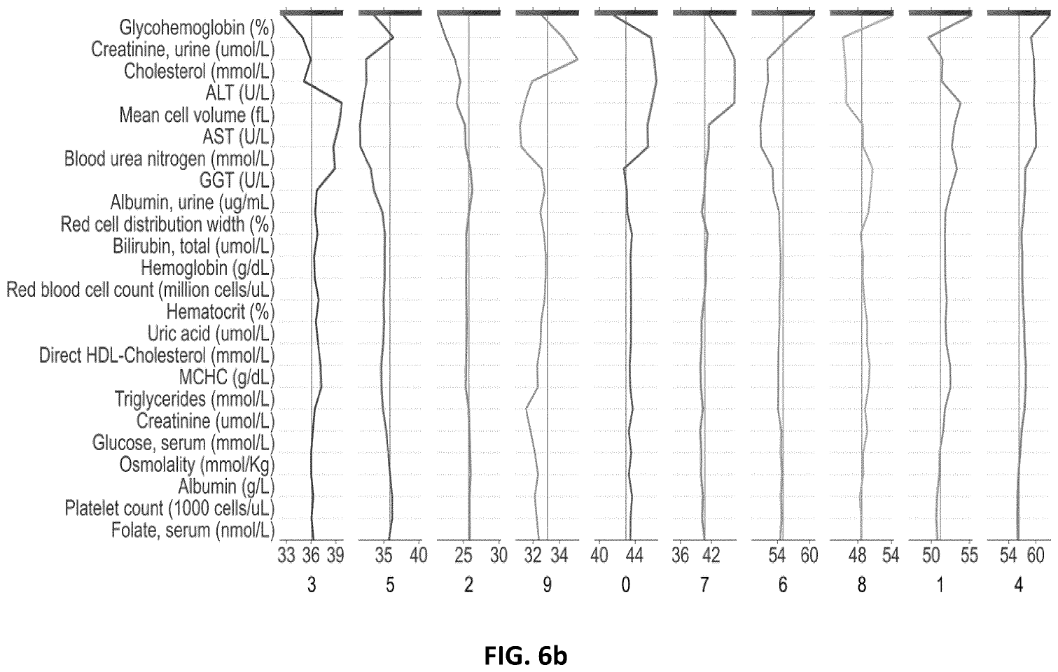

With reference to FIG. 6a is shown the graphical representation of the clustering of the SHAP values obtained for the NHANES dataset (see below), allowing identification of 10 clusters. In FIG. 6b is shown an average individual representative of each cluster: starting from the bottom, the cumulative contribution of each contextualized SHAP value is presented (in positive and negative values) to the predicted final value at the top of the diagram.

It thus appears that the population can be clustered into different groups according to the respective contributions of different biological variables in a difference between the predicted age and the reference.

### EXAMPLES

### NHANES Dataset

A consistent and comprehensive dataset was built in three steps:
(i) all NHANES data from 1999 to 2018 were merged, giving 36,945 variables,
(ii) laboratory variables were selected and aggregated using a dedicated web interface,
(iii) the largest dataset corresponding to the inclusion criteria with the minimum of missing data was defined.

The final dataset included 48 laboratory variables (Table S1) for 60,322 individuals with 30,747 females and 29,575 males, mean age 39.3±19.7 and 39.5±20.2 years old, respectively. The amount of data from 12 to 20 years was twice those of other ages, with a 25% decrease of available subjects from 70 to 79 years old. No major gender imbalance was pointed out across age groups. The amount of missing data was low (25% of individuals with one missing value representing 0.06% of the total values) and uniformly distributed among age and sex They were mainly related to the lack of C-reactive protein, folate, albumin, and creatinine data. In the following steps an imputation method for missing data was implemented, except for XGBoost, able to manage missing data natively.

TABLE S1
List of the 48 biological variables, by alphabetical order

| | Excluded during |
|---|---|
| SAS label | feature selection |
| Albumin (g/L) | |
| Albumin, urine (ug/mL) | |
| Alkaline phosphotase (U/L) | |
| ALT (U/L) | |
| AST (U/L) | |
| Basophils number (1000 cells/uL) | |
| Basophils percent (%) | |
| Bicarbonate (mmol/L) | |
| Bilirubin, total (umol/L) | |
| Blood urea nitrogen (mmol/L) | |
| Cholesterol (mmol/L) | |
| C-reactive protein(mg/dL) | |
| Creatinine (umol/L) | |
| Creatinine, urine (umol/L) | |
| Direct HDL-Cholesterol (mmol/L) | |
| Eosinophils percent (%) | Yes |
| Folate, RBC (nmol/L RBC) | |
| Folate, serum (nmol/L) | |
| GGT (U/L) | |
| Globulin (g/L) | |
| Glucose, serum (mmol/L) | |
| Glycohemoglobin (%) | |
| Hematocrit (%) | |
| Hemoglobin (g/dL) | |
| Iron (umol/L) | Yes |
| LDH (U/L) | Yes |
| Lymphocyte number (1000 cells/uL) | |
| Lymphocyte percent (%) | |
| MCHC (g/dL) | |
| Mean cell hemoglobin (pg) | |
| Mean cell volume (fL) | |
| Mean platelet volume (fL) | Yes |
| Monocyte number (1000 cells/uL) | |
| Monocyte percent (%) | |
| Osmolality (mmol/Kg) | |
| Phosphorus (mmol/L) | |
| Platelet count (1000 cells/uL) | |
| Potassium (mmol/L) | |
| Red blood cell count (million cells/uL) | |
| Red cell distribution width (%) | |
| Segmented neutrophils num (1000 cell/uL) | |
| Segmented neutrophils percent (%) | |
| Sodium (mmol/L) | |
| Total calcium (mmol/L) | |
| Total protein (g/L) | |
| Triglycerides (mmol/L) | |
| Uric acid (umol/L) | |
| White blood cell count (1000 cells/uL) | |

### Selection of the Best and Explainable Algorithm to Define Personalized Physiological Age (PPA)

To define the best and explainable prediction algorithm to define PPA, different machine learning algorithms were assessed using a training and test dataset corresponding to 80% and 20% of the original dataset. To reduce the number of variables and a putative overfitting of the models, variables whose contribution was not statistically greater than chance were eliminated using GrootCV feature selection. Four variables were eliminated: basophils number, mean cell hemoglobin, monocyte number and segmented neutrophils percent, reducing to 44 variables. The choice to keep or not a variable was in part based on redundancy. When another biologically-linked parameters performed better or identically it was kept alone to contribute to PPA. Five classes of machine learning algorithms were then compared for predicting chronological age: tree-based models (Decision Tree, Random Forests and XGBoost), a regularized regression method (ElasticNet, a method with both L1 and L2-norm regularization of the coefficients) and a neural network (MultiLayer Perceptron, MLP).

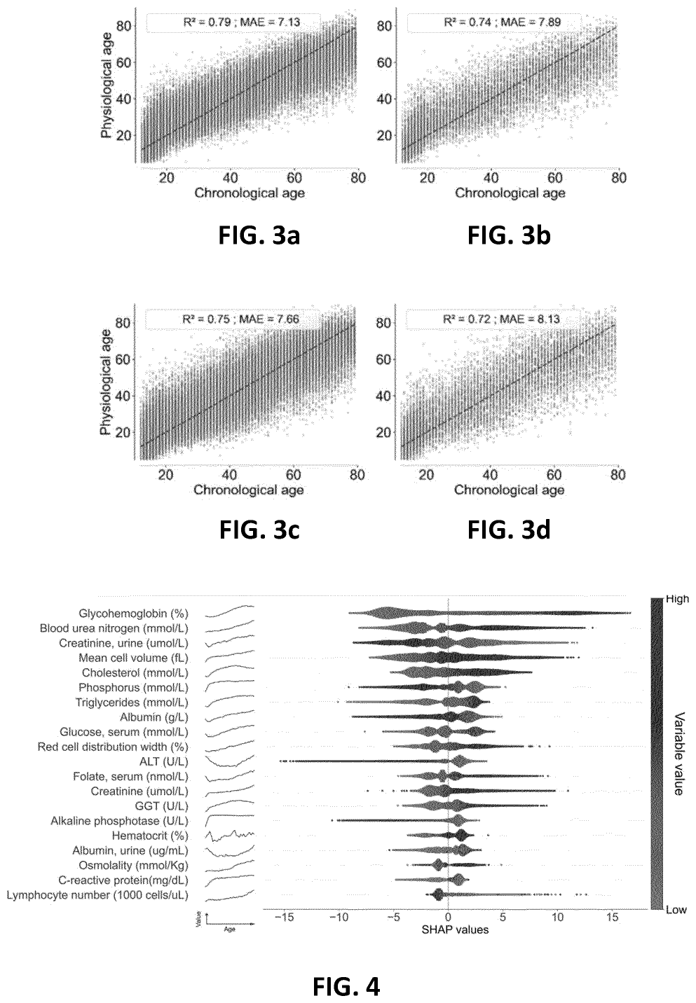

Grid-search exploration of hyper-parameters with a 5-fold cross-validation was performed for each model (Table S2) using the train dataset. Models were evaluated on the basis of their results on the test dataset using R² (coefficient of determination) and MAE (mean absolute error). Regardless of the algorithm classes, similar performances were found on the train and test dataset for both R² and MAE. XGBoost and MLP (multilayer perceptron) achieved the best and similar performances with the lowest standard deviations during cross-validation for XGBoost. Given the high dimensionality (high variables number) and the number of subjects in the database, XGBoost was selected as model for its fastest explainability computation. Error analysis revealed a differential bias of the models to predict age, with a tendency to predict young individuals being older and conversely (FIG. 3a, 3b). To correct bias, custom objective function was introduced during XGBoost training, this greatly minimized bias (FIG. 3c, 3d) while maintaining performance (0.72 and 8.1 on the test dataset for R² and MAE respectively).

### Physiological Age Explainability

To define the contribution of each variable on individual PPA prediction, Shapley Additive exPlanations (SHAP) TreeSHAP framework was used on the XGBoost model with Custom Loss model. The sum of the SHAP values for all variables of the model represents the individual deviation from the mean of chronological age predicted on the entire dataset (39.9 years old in the present model, i.e., the base value). For a given individual, the predicted age was 39.9 plus the sum of all SHAP values. For a set of variables, the higher the overall SHAP value, the more the variable contributes positively to the PPA.

A ranking was performed by the mean absolute value of global SHAP contribution for each variable. From the top-20 variables, many were related to metabolism, whether nitrogenous (e.g., uric metabolites, creatinine), carbonaceous (e.g., glycohemoglobin, triglycerides, glucose), or related to liver function (e.g., albumin, ALT, GGT). Glycohemoglobin appeared as the most contributive parameter (10.7% of the mean total SHAP sum contribution) while serum glucose was ranked 9th. Urinary and blood creatinine, reflecting renal function, were also shown to contribute on PPA prediction. Several parameters directly or indirectly related to erythrocyte, mean cell volume, red cell distribution width, hematocrit, and serum folate, were also distributed among the top-20 variables. Features related to immunity/inflammation (C-reactive protein and lymphocyte number) were ranked 19th and 20th, respectively while other parameters regarding immune system (e.g., monocyte or lymphocyte percent, white blood cell count) had lower impact on SHAP values. The age trend of their mean value usually follows SHAP values (in positive or negative). For example, the mean raw value of glycohemoglobin raises with age, in the same way that increasing its raw value increases its SHAP value. For most of variables (11 variables over 20), the higher the variable value, the higher the deviation from chronological age. No obvious change in explainability profile was found between males and females with similar ranking of variables.

### PPA Contextualized Explainability by Age Groups.

The principle of contextualization is to provide better explainability models by taking as base value the mean prediction of the individuals sharing the same chronological age (instead of the mean prediction of the whole population). In that case, the SHAP contribution of each variable is thus called “contextualized SHAP”. Glycohemoglobin, blood urea nitrogen, mean cell volume and urinary creatinine proved to contribute all along the life course, albeit with a stronger contribution between 40 and 70 years old.

Other variables had more age-specific contributions, such as alkaline phosphatase (12-18 y.o.), ALT and cholesterol (20-40 y.o.) or lymphocyte number and folate (60 y.o. and over). We derived the “iCAD” metric, defined for a given individual as the sum of the contextualized SHAP values.

iCAD Validation and Robustness

Using a multivariate Cox survival model, iCAD was found to be a relevant predictor of mortality (Table 1). Adjusted hazard ratio on gender, chronological age and year of inclusion, indicated that a negative iCAD value was associated to a decreased risk of mortality while non-significant (aHR with 95% Cl of 0.88[0.76;1.03] for the first decile compared to the 5th decile taken as reference). A positive iCAD value was significantly associated to a gradual increase of mortality risk (aHR 95% Cl 1.18[1.01;1.38], 1.37[1.17;1.59], 1.38[1.18;1.60] and 1.69[1.45;1.97] for the 7th to 10th deciles, respectively).

TABLE 1
Validation on mortality data. Adjusted hazard ratio on gender,
chronological and NHANES year of inclusion with 95% confidence
interval were computed according to the iCAD value (sum
of contextualized SHAP values), taken as deciles.

| | aHR [95% CI] | | | |
|---|---|---|---|---|
| iCAD (deciles) | Complete model | | Minimal model | |
| <−11.4 | 0.88 | [0.76; 1.03] | 0.77 | [0.66; 0.89] |
| (−11.4, −7.6] | 0.87 | [0.74; 1.03] | 0.85 | [0.72; 1.0] |
| (−7.6, −4.8] | 0.85 | [0.71; 1.01] | 0.83 | [0.70; 0.98] |
| (−4.8, −2.5] | 1.01 | [0.85; 1.20] | 0.84 | [0.71; 1.0] |
| (−2.5, −0.23] | 1 | | 1 | |
| (−0.23, 2.2] | 1.27 | [1.08; 1.48] | 1.14 | [0.97; 1.33] |
| (2.2, 4.8] | 1.18 | [1.01; 1.38] | 1.17 | [1.0; 1.36] |
| (4.8, 7.8] | 1.37 | [1.17; 1.59] | 1.19 | [1.03; 1.39] |
| (7.8, 12.2] | 1.38 | [1.18; 1.60] | 1.24 | [1.07; 1.44] |
| >12.2 | 1.69 | [1.45; 1.97] | 1.57 | [1.35; 1.83] |
| Gender: Male | 0.64 | [0.60; 0.68] | 0.64 | [0.60, 0.69] |
| Age | 7.76 | [2.09; 28.8] | 7.28 | [1.96, 27.1] |
| Year of inclusion | 1.03 | [0.99; 1.08] | 1.03 | [0.99, 1.07] |
| Age: Year of inclusion | 0.999 | [0.998; 1] | 0.999 | [0.998, 1] |

### Partial Dependence of Contextualized SHAP Values as a New PPA Metric

Partial dependence indicates that the contextualized SHAP contribution for PPA prediction changes according to a variation of the raw variable value (FIG. 5). This relationship for a given variable (the shape of the curves) appeared quite similar between ages, although the amplitude was different. Different types of relationship could be noticed, such as rising sigmoid-like (e.g., glycohemoglobin, blood urea nitrogen), decreasing sigmoid-like (e.g., phosphorus), or a linear tendency (e.g., folate, urinary creatinine). These profiles clearly revealed the different ranges of the variable value for which the corresponding contextualized SHAP values were positive, neutral or negative. For example, while the contextualized SHAP values were negative in low values for glycohemoglobin, a sharp increase occurred in the 5-6% value window. This transition zone, characterized by the passage from zero, is different according to age. Thus, while the threshold of 5.4% seemed to characterize a “normal” range for young subject, it evolved with age, increasing to 5.8% for subjects older than 50. For urinary creatinine, the increase of its value resulted in a decrease of the SHAP contribution, with a value of around 10,000 μmol/L as a null SHAP value. FIG. 5 better reveals a decrease in the normal range of values with age.

### Biological Parameters and Aging

To identify putative specific features at the origin of profiles for individuals, all contextualized SHAP values were clustered, irrespective of chronological age (FIG. 6a, 6b). Clustering highlighted 10 SHAP clusters grouped in two classes according to glycohemoglobin SHAP value. The contribution of low (below clinical threshold at 6%) glycohemoglobin appeared correlated to a “lower” physiological age in older individuals, as in cluster 2. Changes in a reduced set of variables including urinary creatinine, cholesterol, ALT, mean cell volume (MCV), AST, blood urea nitrogen (BUN), and GGT, differentiated the clusters within each class. All other variables weakly contributed to the difference between clusters. FIG. 6b shows the profiles of the variable SHAP-values for each cluster. This suggest that different profiles corresponding to the same iCAD could reflect different physiological ways of aging. Clusters 2 and 4 were characterized by a systematic negative and positive deviation of key biological variables accordingly to a negative and positive deviation from chronological age. All other profiles were characterized by a mix of positive and negative SHAP values of significant variables.

### Generation of a Minimal Model by Recursive Feature Elimination.

In the perspective of a therapeutic use, the best compromise between the PPA estimation exactness and the lowest number of relevant features needed to be pointed out. The results of the run out RFE algorithm showed that 26 variables were sufficient to predict PPA without significantly decreasing the performance of the model estimated by the R².

TABLE S2
List of hyperparameters used during model tuning:

| Model | Grid search parameters | Best hyperparameters found |
|---|---|---|
| Elastic Net | l1_ratio: [0.01, 0.05, 0.1, 0.2, 0.3, 0.4, 0.5, | l1_ratio: 0.99 |
| | 0.6, 0.7, 0.8, 0.9, 0.95, 0.99] | alpha: 0.0001 |
| | alpha: uniform (−4, −2, 0.5) | |
| Random | n_estimators: loguniform (100, 1000) | n_estimators: 598 |
| Forest | max_features: [auto, sqrt] | max_features: auto |
| | max_depth: randint (3, 12) | max_depth: 11 |
| | min_samples_split: [2, 5, 10] | min_samples_split: 5 |
| | min_samples_leaf: [1, 2, 4] | min_samples_leaf: 2 |
| | bootstrap: [True, False] | bootstrap: True |
| Decision | max_depth: int(2, 50) | max_depth: 28 |
| Tree | min_samples_split: int(2, 12) | min_samples_split: 6 |
| | min_samples_leaf: int(2, 50) | min_samples_leaf: 24 |
| Multilayer | n_layers: [2, 3, 4] with hidden_layer_sizes | |
| Perceptron | [16, 32, 64, 128, 256] | n_layers: 2 with |
| | activation: [relu, identity] | hidden_layer_sizes (16, 64, 32, 64) |
| | beta_1: [0.01, 0.05, 0.1, 0.2, 0.3, 0.4, 0.5, | activation: relu |
| | 0.6, 0.7, 0.8, 0.9, 0.95, 0.99] | beta_1: 0.1 |
| | beta_2: [0.01, 0.05, 0.1, 0.2, 0.3, 0.4, 0.5, | beta_2: 0.4 |
| | 0.6, 0.7, 0.8, 0.9, 0.95, 0.99] | alpha: 0.003 |
| | alpha: uniform (−4, −1, 0.5) | |
| XGBoost | max_depth: [3, 4] | max_depth: 3 |
| Model | subsample: uniform(0.2, 0.8, 0.05) | subsample: 0.7 |
| | colsample_bytree: uniform(0.2, 1.0, 0.05) | colsample_bytree: 0.85 |
| | colsample_bylevel: uniform(0.2, 1.0, 0.05) | colsample_bylevel: 0.9 |
| | learning_rate: 10\hat{ }(uniform(−4.0, −1.0, 0.5)) | learning_rate: 0.1 |
| XGBoost | | max_depth: 3 |
| Model with | | subsample: 0.8 |
| custom loss | | colsample_bytree: 1.0 |
| | | colsample_bylevel: 0.5 |
| | | learning_rate: 0.01 |

1. A computer-implemented method for determining a physiological age of a subject, comprising applying, on a set of values comprising at least values of biological variables relative to the subject, a trained model configured to predict the chronological age of a subject based on the set of values, to obtain a predicted age of the subject, wherein the physiological age corresponds to said predicted age.

2. The computer-implemented method according to claim 1, further comprising comparing the physiological age of the subject with the chronological age of the subject, wherein a positive difference between the physiological age of the subject and the chronological age is indicative of premature ageing of the subject.

3. The method according to claim 1, further comprising comparing the physiological age of the subject with a reference age corresponding to a mean age predicted by the trained model on a reference population, and when the physiological age of the subject differs from the reference age, identifying biological variables most contributing to the difference.

4. The computer-implemented method according to claim 3, wherein the reference population is a population of individuals having the same chronological age as the individual.

5. The computer-implemented method according to claim 3, wherein identifying the biological variables most contributing to the difference comprises determining SHAP values associated to each of the biological variables and identifying the SHAP values having highest absolute value.

6. The computer-implemented method according to claim 3, further comprising comparing at least one value of a biological variable most contributing to the difference, to a reference value of said biological variable for the same chronological age.

7. The computer-implemented method according to claim 6, wherein the reference value of a biological variable for a given chronological age is determined as a mean value of the biological variable among a plurality of individuals of said given chronological age for which said biological variable does not contribute to a difference between the predicted age and the chronological age.

8. The computer-implemented method according to claim 3, further comprising determining an ageing profile of the subject among a plurality of pre-established ageing profiles, based on the identified biological or physiological values most contributing to the difference.

9. The computer-implemented method according to claim 8, wherein the plurality of pre-established ageing profiles are determined by:
predicting the chronological age of a plurality of individuals of a population by implementing the trained model, wherein the population comprises for each of a plurality of chronological ages, a plurality of individuals,
determining at least a mean predicted age of the population,
determining, for a plurality of individuals of the population, the SHAP values of the biological variables most contributing to a difference between the predicted age for the individual and the mean predicted age,
performing clustering on the SHAP values to obtain a finite number of clusters, wherein each cluster corresponds to an ageing profile.

10. The computer-implemented method according to claim 1, wherein the trained model is an XGboost model with custom loss function being a function of chronological age.

11. A non-transitory computer-readable storage medium having stored thereon code instructions which, when executed by a processor, cause said processor to implementing a method according to claim 1.

12. A computing system comprising:
a processor,
a non-transitory computer-readable medium storing program code that is executable by the processor,
wherein the processor is configured for executing the program code to perform operations comprising applying, on a set of values of biological variables relative to the subject, a trained model configured to predict the chronological age of a subject based on the set of values, to obtain a predicted age of the subject, wherein the predicted age of the subject corresponds to a physiological age.

13. The computing system according to claim 12, wherein the processor is communicatively coupled via a data network to a client system, and is configured to receive the set of values of biological variables relative to the subject from the client system and to return to the client system the physiological age of the subject, or a difference between the physiological age of the subject and a reference age corresponding to a mean age predicted by the trained model on a population.

14. The computing system according to claim 13, wherein when the computed difference is different from zero, the processor is further configured to compute SHAP values associated to each of the biological variables and identifying the SHAP values having highest absolute value, said SHAP values corresponding to biological variables most contributing to the computed difference.

15. The computing system according to claim 14, wherein the processor is further configured to generate graphical data representing the SHAP values having highest absolute value, wherein the SHAP values contributing to increasing the age predicted by the model with respect to the reference age are represented in a first color and the SHAP values contributing to decreasing the age predicted by the model with respect to the reference age are represented in a second color.
